(19) United States
(12) Patent Application Publication
(10) Pub. No.: US 2026/0008823 A1
(43) Pub. Date: Jan. 8, 2026

(54) CRM197 PROTEIN EXPRESSION METHOD

(71) Applicants: GENOFOCUS CO., LTD., Daejeon (KR); EUBIOLOGICS CO., LTD., Seoul (KR)

(72) Inventors: Jeong Hyun KIM, Gyeonggi-do (KR); Hyun-Do KIM, Gwangju (KR); Eun-Hye PARK, Daejeon (KR); Jung Hyun KANG, Daejeon (KR); Jae Gu PAN, Sejong (KR); Eui Joong KIM, Daejeon (KR); Chankyu LEE, Gangwon-do (KR); Minchul PARK, Gyeonggi-do (KR)

(21) Appl. No.: 19/331,238

(22) Filed: Sep. 17, 2025

Related U.S. Application Data
(63) Continuation of application No. 17/638,822, filed on Feb. 26, 2022.

(30) Foreign Application Priority Data
Sep. 3, 2019 (KR) 10-2019-0108892

(51) Int. Cl. C07K 14/34; C07K 7/08

(52) U.S. Cl. CPC C07K 14/34; C07K 7/08; C07K 2319/02

(57) ABSTRACT

The disclosure relates to a signal sequence for expressing a CRM197 protein in Escherichia coli and secreting same into the periplasm, and use thereof, more specifically, to: a signal sequence for expressing a CRM197 protein; a nucleic acid for coding the signal sequence; a nucleic acid construct or expression vector comprising the nucleic acid and a CRM197 protein gene; a recombinant microorganism having the nucleic acid construct or expression vector introduced therein; and a CRM197 protein production method comprising a step for culturing the recombinant microorganism. A CRM197 protein having the same physicochemical/immunologic properties as the protein isolated from the parent bacteria may be expressed even in Escherichia coli of which a redox potential is not adjusted, and a CRM197 protein having high periplasmic secretion efficiency may be produced even without shifting the pH of a culture medium in order to increase secretion into the periplasm in CRM197 protein production.

### CROSS-REFERENCE TO RELATED APPLICATIONS

This is a continuation under 35 USC § 120 of U.S. patent application Ser. No. 17/638,822 filed Feb. 26, 2022, which in turn is a United States national phase under 35 USC § 371 of International Patent Application No. PCT/KR2019/012899 filed Oct. 2, 2019, which in turn claims priority under 35 USC § 119 of Korean Patent Application No. 10-2019-0108892 filed Sep. 3, 2019. The disclosures of all such applications are hereby incorporated herein by reference in their respective entireties, for all purposes.

### SEQUENCE LISTING

This application includes an electronically submitted sequence listing in .xml format. The .xml file contains a sequence listing entitled “623CON_SeqListing.xml” created on Sep. 16, 2025 and is 77,824 bytes in size. The sequence listing contained in this .xml file is part of the specification and is hereby incorporated by reference herein in its entirety.

### TECHNICAL FIELD

The present invention relates to a signal sequence for expressing a CRM197 protein in E. coli and secreting the CRM197 protein into the periplasm and the use thereof, and more specifically to a signal sequence for expressing a CRM197 protein, a nucleic acid encoding the signal sequence, a nucleic acid construct or expression vector comprising the nucleic acid and a CRM197 protein gene, a recombinant microorganism introduced with the nucleic acid construct or expression vector, and a method for producing the CRM197 protein comprising culturing the recombinant microorganism.

### BACKGROUND ART

Diphtheria toxin (DT) is a proteinaceous exotoxin synthesized and secreted from a pathogenic strain of Corynebacterium diphtheriae. Diphtheria toxin is an ADP-ribosylating enzyme that is secreted as a proenzyme composed of 535 residues and separated into two fragments (fragments A and B) by treatment with a trypsin-like protease. Fragment A is a catalytically active area and is a NAD-dependent ADP-ribosyltransferase that specifically targets the protein synthesis factor EF-2, thus inactivating EF-2 and interrupting protein synthesis in cells.

CRM197 was found through the isolation of various non-toxic forms and partially toxic immunologically cross-reactive forms (CRM or cross-reactive substances) of diphtheria toxin (Uchida et al., Journal of Biological Chemistry 248, 3845-3850, 1973). Preferably, the CRM may have a predetermined size and composition comprising all or part of the DT.

CRM197 is an enzymatically highly inactive and non-toxic form of diphtheria toxin that contains a single amino acid substitution (G52E). This mutation imparts intrinsic flexibility to the active-site loop located in front of the NAD-binding site, thereby lowering the binding affinity of CRM197 to NAD and removing the toxicity of DT (Malito et al., Proc. Natl. Acad. Sci. USA 109 (14):5229-342012). CRM197, like DT, has two disulfide bonds. One disulfide bond links Cys186 to Cys201 to thereby link fragment A to fragment B. The other disulfide bond links Cys461 to Cys471 in fragment B. DT and CRM197 have nuclease activity derived from fragment A (Bruce et al., Proc. Natl. Acad. Sci. USA 87, 2995-8, 1990).

A number of antigens have low immunogenicity, especially in infants and young children, unless chemically linked to proteins, and are thus produced into conjugates or conjugate vaccines. In these conjugate vaccines, the protein component is also referred to as a “carrier protein”. CRM197 is commonly used as a carrier protein in the protein-carbohydrate conjugation and hapten-protein conjugation. CRM197, as a carrier protein, has several advantages over diphtheria toxoid as well as other toxoid proteins (Shinefield Vaccine, 28:4335, 2010).

Methods for preparing diphtheria toxin (DT) are well known in the art. For example, DT may be produced by purification of the toxin from a culture of Corynebacterium diphtheriae, followed by chemical detoxification, or by purification of a recombinant or genetically detoxified analog of the toxin.

The abundance of proteins made it impossible to realize mass production of diphtheria toxins such as CRM197 for use in vaccines. This problem has previously been addressed by expression of CRM197 in E. coli (Bishai, et al., J. Bacteriol. 169:5140-5151), and Bishai et al. have reported a recombinant fusion protein containing a toxin (including tox signal sequence) leading to the production of degraded proteins.

Compared to cytoplasmic production, the production of bacterial toxins in the periplasm is characterized in that i) the protein is produced in a mature form after cleavage of the signal peptide, or ii) the periplasm of E. coli is an oxidizing environment that allows the formation of disulfide bonds, which can aid in the production of soluble, properly folded proteins, iii) the periplasm of E. coli contains less protease than the cytoplasm, which helps avoid proteolytic cleavage of expressed proteins, and iv) the periplasm also contains fewer proteins, which allows a recombinant protein to be obtained with higher purity.

In general, the presence of signal sequences on proteins facilitates transport (prokaryotic hosts) or secretion (eukaryotic hosts) of the proteins into the periplasm. In the prokaryotic hosts, the signal sequences coordinate newly formed proteins to the periplasm across the inner membrane and then are cleaved. That is, it is important to search for signal sequences capable of more efficiently mass-producing commercially essential proteins, and it is necessary to develop recombinant microorganisms.

Accordingly, as a result of extensive efforts to develop a method for producing the CRM197 protein in an efficient and cost-effective manner, the present inventors selected specific signal sequences, designed nucleotide sequences by combining the codon context with a secondary structure so as to optimize translation in E. coli, optimized expression in E. coli of CRM197 nucleotide sequences encoding the CRM197 protein, and found that CRM197 was efficiently expressed in E. coli and was efficiently secreted into the periplasm without pH change when these were used, thus completing the present invention.

The information disclosed in this Background section is provided only for better understanding of the background of the present invention, and therefore it may not include information that forms the prior art that is already obvious to those skilled in the art.

### SUMMARY OF THE INVENTION

It is one object of the present invention to provide a signal sequence for expressing a CRM197 protein having a specific sequence, a nucleic acid encoding the signal sequence, and a method of producing a CRM197 protein using the nucleic acid in order to maximize expression of the CRM197 protein while minimizing the toxicity of the CRM197 protein to E. coli by secreting the CRM197 protein into the periplasm of E. coli.

In order to accomplish the objects, the present invention provides a signal sequence for expressing a CRM197 protein, represented by any one of amino acid sequences of SEQ ID NO: 13 to SEQ ID NO: 21.

The present invention also provides a nucleic acid encoding the signal sequence for expressing the CRM197 protein.

The present invention also provides a nucleic acid construct or expression vector comprising the nucleic acid and a gene of the CRM197 protein, a recombinant microorganism introduced with the nucleic acid construct or expression vector, and a method for producing a CRM197 protein comprising culturing the recombinant microorganism.

### BRIEF DESCRIPTION OF DRAWINGS

FIG. 1 is a schematic diagram illustrating an expression module, TPB1Tv1.3, wherein Ptrc means a tre promoter, RBS means A/U rich enhancer+SD, λtR2 & T7Te, and rrnB T₁T₂ mean transcription terminators, and BsaI and DraI mean restriction enzyme sites used for cloning.

FIG. 2 illustrates an E. coli expression plasmid pHex1.3, wherein Ori (pBR322) means the origin of replication of pBR322, KanR means a kanamycin marker, lacI means a lacI gene, and the other symbols are the same as in FIG. 1.

FIG. 3 is a schematic diagram illustrating production of the CRM197 expression plasmid, wherein T1 means a λtR2 & T7Te transcription terminator, T2 means rrnB T₁T₂ transcription terminators, arrows mean PCR primers, the alphabetic characters A, B, C and D mean homologous regions for LIC, and the remaining symbols are the same as in FIG. 1.

FIG. 4 illustrates the expression behaviors of L3 and L5 fusion CRM197 in various E. coli strains, and results of Coomassie staining (left) and Western blotting (right) after SDS-PAGE of total E. coli cells, wherein C means C2894H, B means BL21 (DE3), W means W3110-1, O means Origami™ 2, S means a shuffle, C-v represents C2984H containing pHex1.3 used as a negative control, L3 and L5 represent fused signal sequences, and CRM197 represents reference CRM197, and cells are loaded in each well at a density corresponding to OD₆₀₀ of 0.025 for Coomassie staining and at a density corresponding to OD₆₀₀ of 0.0005 for Western blotting.

FIG. 5 illustrates the effects of the type and concentration of an inducer on the expression of CRM197 induced by L5, and illustrates the cases of Coomassie staining (left) and Western blotting (right) after SDS-PAGE, wherein the amounts of loaded cells are the same as in FIG. 4, I means an insoluble fraction, S means a soluble fraction, (A) shows culture at 25° C., and (B) shows culture at 30° C.

FIG. 6 illustrates the position of a CRM197 protein induced by L5 fusion, wherein the top shows Coomassie staining after SDS-PAGE, the bottom shows Western blotting, Cr represents reference CRM197, the arrow represents the position of matured CRM197, P1 means a supernatant obtained after treatment with plasma membrane induction buffer, P2 means a periplasmic fraction, and Cy means a cytoplasm fraction.

FIG. 7 illustrates the effects of the type and concentration of the inducer on the expression of CRM197 induced by L3, wherein (A) shows culture at 25° C. and (B) shows culture at 30° C.

FIG. 8 shows the position of the CRM197 protein induced by L3 fusion.

FIG. 9 shows changes in pH of the L3 strain, temperature, impeller speed and dissolved oxygen (DO), and the addition time of the expression inducer, wherein (A) shows the case in which the temperature is maintained at 30° C., and (B) shows the case in which the temperature is lowered to 25° C. before expression induction.

FIG. 10 shows changes in pH of the L5 strain, temperature, impeller speed and dissolved oxygen (DO) and the addition time of the expression inducer.

FIG. 11 shows the change in CRM197 protein expression before and after addition of the expression inducer during culture, wherein (A) shows expression of the L3 strain induced at 30° C., (B) shows expression of the L3 strain induced at 25° C., (C) shows expression of the L5 strain induced at 25° C. 200 ng of reference CRM197 was loaded in lines 3 and 10 in (A), lines 4 and 10 in (B), and lines 3 and 9 in (C). Lines 1 and 2 in (A), lines 1, 2 and 3 in (B), and lines 1 and 2 in (C) are cell culture solutions before addition of the expression inducer and line 16 in (A) and line 16 (C) are supernatants after completion of culture, the subsequent lines are cell culture solutions sampled every 2 hours after addition of the inducer. The amount of loading is the same as in FIG. 4.

FIG. 12 illustrates the expression and separation behaviors of CRM197 protein in L3/L5 strains, wherein (A) illustrates the protein separation behavior in culture using L3, (B) illustrates the protein separation behavior in culture using L5, Line 1 was loaded with 200 ng of reference CRM197, line 2 is the total cell culture solution after completion of culture, line 3 is the supernatant after treatment with plasma membrane induction buffer, lines 4 and 7 are periplasmic fractions (line 7 being loaded with 4 times as much protein as line 4) and lines 5 and 6 represent cytoplasm fractions.

FIG. 13 illustrates the result of SDS-PAGE of samples purified through DEAE chromatography (A) and HA chromatography (B). In (A), line 1 means CRM197 produced by Corynebacterium, line 2 means the protein present in the recovered periplasmic fraction, line 3 means the sample before loading in DEAE chromatography, which is concentrated twice after ultrafiltration, lines 4 and 5 are used to detect impurity proteins excluding CRM197 using a flow-through mode of DEAE chromatography and washing solutions, line 6 is an eluted sample from which impurities have been removed, line 7 is a sample eluted using a high-concentration salt to remove all proteins bound to the resin. HA Elu in (B) is CRM197 eluted after removing the protein not bound to the HA resin.

FIG. 14 illustrates the result of SEC-HPLC analysis of the final purified CRM197, wherein the purity was 99% or more.

FIG. 15 shows results of SDS-PAGE (left) and Western blot (right). Line 1 is CRM197 produced by Corynebacterium and line 2 is CRM197 produced by E. coli (L3). Both CRM197 showed bands at the same location.

FIG. 16 illustrates the result of intact protein molecular mass analysis using LC/MS, wherein the molecular weight was 58,409 Da, which corresponds to the theoretical molecular weight.

FIG. 17 illustrates the result of circular dichroism (CD) analysis and indicates that there is no difference in higher-order structure between CRM197 (⋅) produced by Corynebacterium and CRM197 (X) produced by E. coli pHex-L3.

FIG. 18 illustrates the result of fluorescence spectrum analysis, wherein CRM197 (⋅) produced by Corynebacterium and CRM197 (solid line) produced by E. coli pHex-L3 had the same maximum emission wavelength of 338 nm.

### DETAILED DESCRIPTION AND PREFERRED EMBODIMENTS OF THE INVENTION

Unless defined otherwise, all technical and scientific terms used herein have the same meanings as appreciated by those skilled in the field to which the present invention pertains. In general, the nomenclature used herein is well-known in the art and is ordinarily used.

In one embodiment of the present invention, 9 signal sequences were fused with a CRM197 protein to induce expression thereof. For each signal sequence, nucleotide sequences (SEQ ID NO: 4 to SEQ ID NO: 12) optimized for translation were designed in consideration of the codon context and the secondary structure of mRNA. These constructs were inserted into the expression plasmid pHex1.3, and the expression of CRM197 was observed in five E. coli strains to find the optimal E. coli strain for each construct. In addition, the culture temperature and the type and concentration of the inducer were set in the selected E. coli. As a result of transforming various E. coli strains with the constructs, the CRM197 protein could be expressed in a soluble form even in strains in which the genes (trxB, gor) related to the redox potential are not engineered, and could be secreted into the periplasm.

Thus, in one aspect, the present invention is directed to a signal sequence for expressing a CRM197 protein, represented by any one of amino acid sequences of SEQ ID NO: 13 to SEQ ID NO: 21.

In another aspect, the present invention is directed to a nucleic acid encoding the signal sequence for expressing the CRM197 protein.

In the present invention, the nucleic acid may be represented by any one of nucleotide sequences of SEQ ID NO: 4 to SEQ ID NO: 12, preferably represented by a nucleotide sequences of SEQ ID NO: 6 or SEQ ID NO: 8, but is not limited thereto.

As used herein, the term “signal sequence for expressing a CRM197 protein” means a signal sequence for expression of a CRM197 protein and secretion of the CRM197 protein into the periplasm.

In one embodiment of the present invention, the signal sequence of the protein targeted to the outer membrane of E. coli and the signal sequence derived from the M13 phage were selected (Table 3) in order to secrete the CRM197 protein into the periplasm. The nucleotide sequences (SEQ ID NO: 4 to SEQ ID NO: 12) were designed by combining the codon context with a secondary structure in order to optimize translation of the selected signal sequence in E. coli.

In another aspect, the present invention is directed to a nucleic acid construct comprising the nucleic acid encoding the signal sequence for expression of a CRM197 protein and a gene of the CRM197 protein.

In another aspect, the present invention is directed to an expression vector comprising the nucleic acid encoding the signal sequence for expressing a CRM197 protein and a gene of the CRM197 protein.

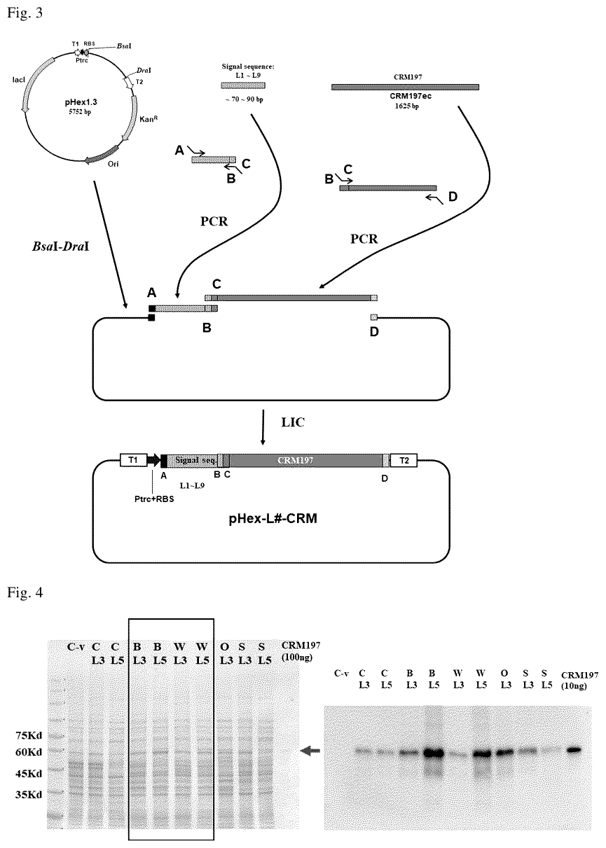

In one embodiment of the present invention, the DNA sequence coding the amino acid sequence of the CRM197 protein (SEQ ID NO: 3) was also optimized for E. coli expression (SEQ ID NO: 2). The DNA fragment of each designed signal sequence and the optimized CRM197 DNA fragment were inserted into the plasmid pHex1.3 (FIG. 3).

In the present invention, any CRM197 protein gene may be used without limitation, as long as it is a gene encoding a CRM197 protein. Preferably, the CRM197 protein gene may be represented by the nucleotide sequence of SEQ ID NO: 2, but is not limited thereto.

As used herein, the term “transformation” means introduction of a specific external DNA strand from outside the cells into the cells. A host microorganism comprising the introduced DNA strand is referred to as a “transformed microorganism”. As used herein, the term ‘transformation’ meaning introducing DNA into a host and making the DNA replicable by an extrachromosomal factor or chromosomal integration indicates that a vector comprising a polynucleotide encoding a target protein is introduced into a host cell, or the polynucleotide encoding the target protein is integrated into the chromosome of the host cell to express the protein encoded by the polynucleotide in the host cell. The transformed polynucleotide comprises both a transformed polynucleotide inserted into and located inside the chromosome of the host cell and a transformed polynucleotide located outside the chromosome, so long as it can be expressed in the host cell.

As used herein, the term “nucleic acid construct” comprises both a nucleic acid construct inserted into and located inside the chromosome of the host cell and a nucleic acid construct located outside the chromosome, so long as it can be expressed in the host cell.

In addition, as used herein, the term “polynucleotide” is used interchangeably with the term “nucleic acid” and comprises DNA and RNA encoding a target protein. The polynucleotide may be introduced in any form so long as it can be introduced into a host cell and expressed therein. For example, the polynucleotide may be introduced into the host cell in the form of an expression cassette, which is a gene construct comprising all of the elements necessary for self-expression. The expression cassette typically comprises a promoter, a transcription termination signal, a ribosome-binding site and a translation termination signal, which is operably linked to the nucleic acid. The expression cassette may take an expression vector allowing for self-replication. The polynucleotide may also be introduced into the host cell in its native form and be operably linked to a sequence necessary for expression in the host cell.

As used herein, the term “vector” means a DNA product comprising a DNA sequence operably linked to a suitable regulatory sequence capable of expressing the DNA in a suitable host. The vectors may be plasmids, phage particles, or simple potential genomic inserts. When transformed into a suitable host, vectors may be replicated or perform functions independent of the host genomes, or some thereof may be integrated with the genomes. Plasmids are currently the most commonly used form of vector. Thus, the terms “plasmid” and “vector” are used interchangeably.

In consideration of the objects of the present invention, use of a plasmid vector is preferred. Typical plasmid vectors that can be used to accomplish the objects comprise (a) a replication origin to efficiently conduct replication so as to comprise several or several hundreds of plasmid vectors in each host cell, (b) an antibiotic resistance gene to screen host cells transformed with plasmid vectors, and (c) a restriction enzyme cleavage site into which a foreign DNA fragment is inserted. Even if an appropriate restriction enzyme cleavage site is not present, the vector and foreign DNA can be easily ligated using a synthetic oligonucleotide adaptor or a linker according to a conventional method.

Furthermore, when the gene is aligned with another nucleic acid sequence based on a functional relationship therebetween, it is said to be “operably linked” thereto. This may be gene(s) and regulatory sequence(s) linked in such a way so as to enable gene expression when a suitable molecule (e.g., a transcriptional activator protein) is linked to the regulatory sequence(s). For example, DNA for a pre-sequence or secretory leader is operably linked to DNA for a polypeptide when expressed as a preprotein involved in the secretion of the polypeptide; a promoter or enhancer is operably linked to a coding sequence when it affects the transcription of the sequence; a ribosome-binding site is operably linked to a coding sequence when it affects the transcription of the sequence; or the ribosome-binding site is operably linked to a coding sequence when positioned to facilitate translation.

Generally, the term “operably linked” means that the linked DNA sequence is in contact, or that a secretory leader is in contact therewith and is present in the reading frame. However, the enhancer need not be in contact therewith. The linkage of these sequences is carried out by ligation at convenient restriction enzyme sites. When no such site exists, a synthetic oligonucleotide adaptor or a linker according to a conventional method is used.

In the present invention, the expression vector may further comprise a Trc promoter.

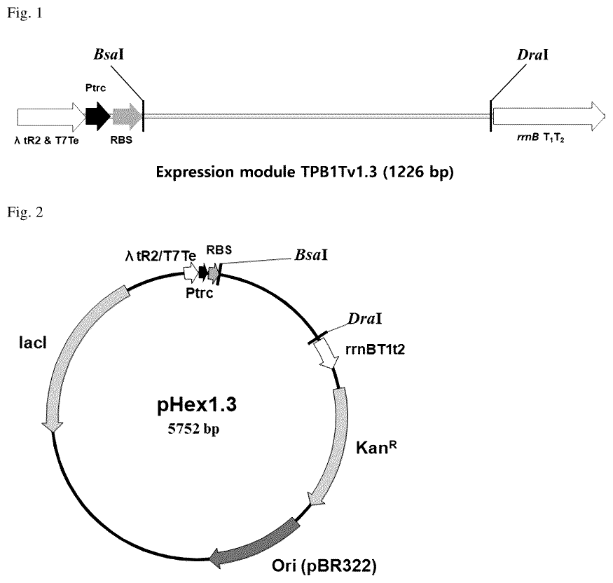

In the present invention, the expression vector may be pHex 1.3, but is not limited thereto. CRM197 is known to be highly toxic to E. coli due to the nuclease activity thereof. Therefore, CRM197 expression under undesired conditions can adversely affect E. coli growth. The E. coli expression plasmid pHex1.3 has the LacI gene and can suppress background expression of the tre promoter, and the expression module TPB1Tv1.3 (FIG. 1) inserted into this plasmid is designed to suppress the expression of CRM197 transcribed from the plasmid-derived promoter by inserting a 2 phage-derived tR2 transcription terminator and a T7 phage-derived Te transcription terminator into the upstream of the CRM197 gene to be expressed, and an E. coli-derived rrnB T1T2 transcription terminator into the downstream thereof.

In another aspect, the present invention is directed to a recombinant microorganism introduced with the nucleic acid construct or the expression vector.

In the present invention, the recombinant microorganism may be Escherichia coli, but is not limited thereto.

As the recombinant microorganism, host cells having high DNA introduction efficiency and high expression efficiency of the introduced DNA are commonly used, all of bacteria, yeast, mold, etc., that is, all microorganisms including prokaryotic and eukaryotic cells, are available, and in the example of the present invention, E. coli was used, but the present invention is not limited thereto, and any type of microorganism may be used as long as the CRM197 protein can be sufficiently expressed.

It should be understood that not all vectors function identically in expressing the DNA sequences of the present invention. Likewise, not all hosts function identically for the same expression system. However, those skilled in the art will be able to make appropriate selections from among various different vectors, expression regulatory sequences and hosts without excessive burden of experimentation and without departing from the scope of the present invention. For example, selection of a vector should be carried out in consideration of the host because the vector should be replicated therein. The number of times the vector replicates, the ability to control the number of times the vector replicates, and the expression of other proteins encoded by the corresponding vector, such as the expression of antibiotic markers, should also be considered.

The transformed recombinant microorganism may be prepared according to any known transformation method.

In the present invention, a method for inserting the gene into the chromosome of host cells may be selected from conventionally known genetic manipulation methods, for example, methods using retroviral vectors, adenovirus vectors, adeno-associated viral vectors, herpes simplex viral vectors, poxvirus vectors, lentiviral vectors, or non-viral vectors.

Also, the transformation may be performed by directly inserting the nucleic acid construct into the chromosome of host cells, in addition to using expression vectors.

In general, electroporation, lipofection, ballistic delivery, virosomes, liposomes, immunoliposomes, polycations or lipid: nucleic-acid conjugates, naked DNA, artificial virions, chemically promoted DNA influx, calcium phosphate (CaPO₄) precipitation, calcium chloride (CaCl₂) precipitation, microinjection, a lithium acetate-DMSO method, etc. may be used.

Sonoporation, for example, methods using a Sonitron 2000 system (Rich-Mar), may also be used for delivery of nucleic acids, and other representative nucleic acid delivery systems include Amaxa Biosystems (Cologne, Germany), Maxcyte, Inc. (Rockville, Maryland) and BTX Molecular System (Holliston, Mass.). Lipofection methods are disclosed in U.S. Pat. Nos. 5,049,386, 4,946,787, and 4,897,355, and lipofection reagents are commercially available, for example, TRANSFECTAM™ and LIPOFECTIN™. Cationic or neutral lipids suitable for effective receptor-recognition lipofection of polynucleotides include Felgner's lipids (WO91/17424 and WO91/16024), which may be delivered to cells through ex-vivo transduction and to target tissues through in-vivo transduction. Methods for preparing a lipid: nucleic-acid complex containing a target liposome, such as an immunolipid complex, are well known in the art (Crystal, Science., 270:404-410, 1995; Blaese et al., Cancer Gene Ther., 2:291-297, 1995; Behr et al., Bioconjugate Chem., 5:382389, 1994; Remy et al., Bioconjugate Chem., 5:647-654, 1994; Gao et al., Gene Therapy., 2:710-722, 1995; Ahmad et al., Cancer Res., 52:4817-4820, 1992; U.S. Pat. Nos. 4,186,183; 4,217,344; 4,235,871; 4,261,975; 4,485,054 4,501,728; 4,774,085; 4,837,028; 4,946,787).

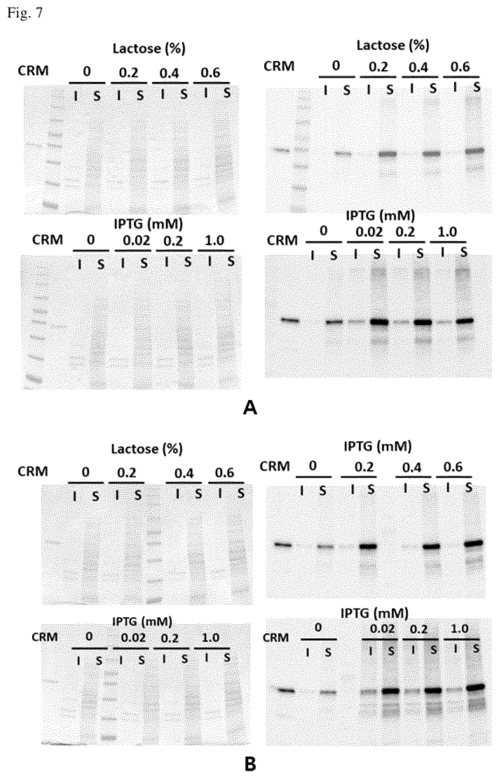

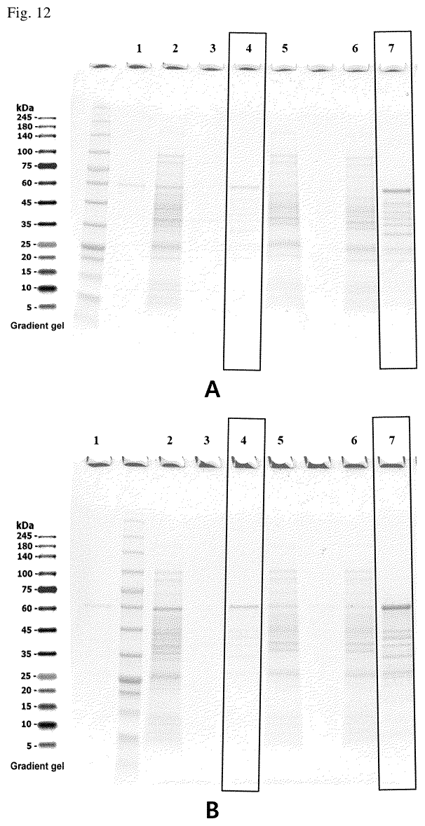

In one embodiment of the present invention, as a result of transformation of various E. coli strains with the prepared plasmid vector (Table 4), for the L3 fusion, CRM197 was expressed in all strains, and for the LS fusion, CRM197 was expressed in strains excluding Origami™ 2 (FIG. 4). Both the L3 and L5 fusions were capable of expressing the CRM197 protein in a soluble form even in strains in which the genes (trxB, gor) related to redox potential were not engineered, and the protein had the same physicochemical/immunological properties as the protein isolated from the parent strains. In addition, in the case of L3 fusion, CRM197 protein could be expressed in a soluble form at 25° C. as well as at 30° C. (FIGS. 7 and 12A).

The previous report showed that when cultured at a pH of 6.5 to 6.8 and then shifted to pH 7.5 during induction, secretion of CRM197 into the periplasm is improved. In contrast, the strain prepared in the present invention was found to efficiently secrete CRM197 into the periplasm without a pH shift of the medium (FIG. 12). In the case of L5 fusion in high-concentration culture without pH shifting, the productivity of CRM197 was 3.7 g/L, and 2 g/L or more of CRM197 was secreted into the periplasm.

In another aspect, the present invention is directed to a method for producing a CRM197 protein comprising (a) culturing a recombinant microorganism introduced with the nucleic acid construct or the expression vector to produce a CRM197 protein, and (b) recovering the produced CRM197 protein.

In the present invention, step (b) may comprise recovering the CRM197 protein secreted into the periplasm.

Hereinafter, the present invention will be described in more detail with reference to examples. However, it will be obvious to those skilled in the art that these examples are provided only for illustration of the present invention and should not be construed as limiting the scope of the present invention.

### EXAMPLE 1: PREPARATION of CRM197 OVEREXPRESSION PLASMID

### Example 1.1: Construction of Plasmid pHex1.3

An E. coli expressing plasmid, pHex1.3, was prepared as follows. After double digestion of ptrc99a (Amann et al., Gene. 69, 301-15, 1988) with SspI and DraI, an about 3.2 kb DNA fragment was purified using agarose electrophoresis. The kanamycin resistant gene was amplified using PCR. The template used herein was plasmid pCR2.1, and the primers used herein were KF2 and KR (Table 1).

TABLE 1
PCR primers

| | Nucleotide | sequence | | SEQ |
|---|---|---|---|---|
| Primer | (5′ -> 3′) | Template | Purpose | ID NO |
| KF2 | GCGGATCCAAGAGACAGGA | pCR2.1 | Amplification | 22 |
| | TGAGGATCGTTTCGC | | of Km gene | |
| KR | CGGATATCAAGCTTGGAAA | | | 23 |
| | TGTTGAATACTCATACTCT | | | |
| | TC | | | |
| TPB_F | GAGATCCGGAGCTTATACT | TPB1Tv1.3 | Amplification | 24 |
| | GAGCTAATAAC | | of TPB1Tv1.3, | |
| TPB_R | GAAAAATAAACAAAAACAA | | identification | 25 |
| | AAAGAGTTTG | | of insert | |
| pHex_F | TACAAACTCTTTTTGTTTT | pHex1.1 | Amplification | 26 |
| | TGTTTATTTTTC | | of pHex1.1 | |
| pHex_R | CCTGTTATTAGCTCAGTAT | | backbone | 27 |
| | AAGCTCCGGATCTCG | | | |
| L1F | AATTGGAGGAACAATATGA | L1 | Amplification | 28 |
| | AATATCT | | of L1 signal | |
| L1R | AACATCGTCAGCGCCCGCC | | seq. | 29 |
| | ATCGCCGGCT | | | |
| L2F | TTGGAGGAACAATATGAAA | L2 | Amplification | 30 |
| | AAAAGCCT | | of L2 signal | |
| L2R | AACATCGTCAGCGCCGGCA | | seq. | 31 |
| | AACGACAGCAT | | | |
| L3F | TTGGAGGAACAATATGCGT | L3 | Amplification | 32 |
| | TCTGTGA | | of L3 signal | |
| L3R | AACATCGTCAGCGCCGGCG | | seq. | 33 |
| | CTCACGCAA | | | |
| L4F | TTGGAGGAACAATATGCGT | L4 | Amplification | 34 |
| | GCGAAACT | | of L4 signal | |
| L4R | AACATCGTCAGCGCCGGCA | | seq. | 35 |
| | AAGCTGGAAAT | | | |
| L5F | TTGGAGGAACAATATGAAA | L5 | Amplification | 36 |
| | AAAACC | | of L5 signal | |
| L5R | AACATCGTCAGCGCCCGCC | | seq. | 37 |
| | TGCGCCACGGT | | | |
| L6F | TTGGAGGAACAATATGAAA | L6 | Amplification | 38 |
| | CTGCTGA | | of L6 signal | |
| L6R | AACATCGTCAGCGCCGGCA | | seq. | 39 |
| | AAACTACTGCT | | | |
| L7F | TTGGAGGAACAATATGAAA | L7 | Amplification | 40 |
| | AAACTG | | of L7 signal | |
| L7R | ACATCGTCAGCGCCGCTGT | | seq. | 41 |
| | GGCTGTAAAA | | | |
| L8F | TTGGAGGAACAATATGAAA | L8 | Amplification | 42 |
| | GCGACGAAA | | of L8 signal | |
| L8R | AACATCGTCAGCGCCGCCC | | seq. | 43 |
| | GCCAGCAGCGT | | | |
| L9F | TTGGAGGAACAATATGAAA | L9 | Amplification | 44 |
| | GGTCTGAA | | of L9 signal | |
| L9R | AACATCGTCAGCGCCCGCA | | seq. | 45 |
| | TGACCCGCGCA | | | |
| C1F | AGCCGGCGATGGCGGGCGC | CRM197ec | Amplification | 46 |
| | TGACGATG | | of CRM197 | |
| | | | compatible | |
| | | | with L1 | |
| C2F | ATGCTGTCGTTTGCCGGCG | CRM197ec | Amplification | 47 |
| | CTGACGATG | | of CRM197 | |
| | | | compatible | |
| | | | with L2 | |
| C3F | TTGCGTGAGCGCCGGCGCT | CRM197ec | Amplification | 48 |
| | GACGATG | | of CRM197 | |
| | | | compatible | |
| | | | with L3 | |
| C4F | TTTCCAGCTTTGCCGGCGC | CRM197ec | Amplification | 49 |
| | TGACGATG | | of CRM197 | |
| | | | compatible | |
| | | | with L4 | |
| C5F | ACCGTGGCGCAGGCGGGCG | CRM197ec | Amplification | 50 |
| | CTGACGATG | | of CRM197 | |
| | | | compatible | |
| | | | with L5 | |
| C6F | AGCAGTAGTTTTGCCGGCG | CRM197ec | Amplification | 51 |
| | CTGACGATG | | of CRM197 | |
| | | | compatible | |
| | | | with L6 | |
| C7F | TTTTACAGCCACAGCGGCG | CRM197ec | Amplification | 52 |
| | CTGACGATG | | of CRM197 | |
| | | | compatible | |
| | | | with L7 | |
| C8F | TGCTGGCGGGCGGCGCTGA | CRM197ec | Amplification | 53 |
| | CGATG | | of CRM197 | |
| | | | compatible | |
| | | | with L8 | |
| C9F | CGCGGGTCATGCGGGCGCT | CRM197ec | Amplification | 54 |
| | GACGATG | | of CRM197 | |
| | | | compatible | |
| | | | with L9 | |
| C9R | GATATCCGCTTTTCATTAG | CRM197ec | Reverse primer | 55 |
| | CTTTTAATCTCGAAGAA | | for | |
| | | | amplification | |
| | | | of all CRM197 | |
| crm_mconf | GGCGCAAGCGTGCGCGGGT | pHex-L#- | Identification | 56 |
| | AACCGTGTGCG | CRM | of insert | |

The PCR reaction solution was prepared using 2.5 mM of each dNTP, 10 pmol of each primer, 200 to 500 ng of template DNA, 1.25 U of PrimeSTAR HS DNA Polymerase (Takara Bio Inc., Japan), and 50 μl of a reaction volume, and PCR was performed for 30 cycles, each comprising three steps at 98° C. for 10 seconds, at 60° C. for 5 seconds, and at 72° C. min/kb. After double digestion of about 0.8 kb DNA fragment produced under PCR conditions with BamHI and HindIII, the DNA fragment was filled-in with a klenow fragment to form a blunt end and then was ligated with the 3.2 kb DNA fragment prepared in the previous process using T4 DNA ligase. This reaction solution was transformed into E. coli C2984H to prepare pHex 1.1 in which the selection marker was substituted from Amp to Km.

The expression module TPB1Bv1.3, comprising promoters, RBS, and transcription terminators (FIG. 1), was synthesized in Bioneer (SEQ ID NO: 1). Process of loading the expression module TPB1Bv1.3 on pHex1.3 is as follows. A DNA fragment (1255 bp) obtained by PCR amplification using the expression module TPB1Bv1.3 as a template and TPB_F and TPB_R as primers (Table 1) was assembled in vitro with a DNA fragment (4561 bp) obtained through PCR amplification using pHex 1.1 as a template and pHex_F and pHex_R as primers via ligation-independent cloning (LIC; Jeong et al., Appl Environ Microbiol. 78, 5440-3, 2012) under the conditions shown in Table 2, and the resulting product was transformed into E. coli C2984H to prepare pHex1.3 (FIG. 2).

TABLE 2
LIC reaction solution

| | Stock conc. |
|---|---|
| Linearized vector | 100 ng |
| Insert 1 | 40 ng |
| Insert 2 (if necessary) | 40 ng |
| T4 DNA polymerase(NEB) | 1U |
| H₂O | Up to 10 μL |

LIC reaction conditions: The vectors were digested with restriction enzymes or were produced into linear DNA fragments through PCR. Inserts were prepared using PCR. The vectors were mixed with the inserts as shown in the table above, and then reacted at room temperature for 2 minutes and 30 seconds.

### Example 1.2: Construction of CRM197 Gene

The nucleotide sequence (CRM197ec) of CRM197 optimally expressed in E. coli was synthesized in GenScript (SEQ ID NO: 2). The amino acid sequence coded by CRM197ec is SEQ ID NO: 3.

### Example 1.3: Construction of Signal Sequence Gene

The signal sequences used to secrete the CRM197 protein into the periplasm of E. coli are shown in Table 3 below (SEQ ID NOS: 13 to 21). To optimize expression in E. coli, the DNA of SEQ ID NOS: 4 to 12 was synthesized in consideration of the codon context and secondary structure (Table 3).

TABLE 3
Signal sequences used in present invention

| | | Amino acid | Nucleotide sequence |
|---|---|---|---|
| Name | Description | sequence | (5′ -> 3′) |
| L1 | PelB signal | MKYLLPTAAAGLLLL | GGTCTCATATGAAATATCTGTT |
| | sequence | AAQPAMA | ACCGACCGCCGCTGCCGGACTG |
| | | (SEQ ID NO: 13) | CTGTTACTGGCGGCGCAGCCGG |
| | | | CGATGGCGGGCGAGAGACC |
| | | | (SEQ ID NO: 4) |
| L2 | M13 G8 signal | MKKSLVLKASVAVAT | GGTCTCATATGAAAAAAAGCCT |
| | sequence | LVPMLSFA | GGTTCTGAAAGCGTCTGTTGCG |
| | | (SEQ ID NO: 14) | GTGGCGACGCTGGTGCCGATGC |
| | | | TGTCGTTTGCCGGCGAGAGACC |
| | | | (SEQ ID NO: 5) |
| L3 | E. coli | MRSVIVAFLFACSFC | GGTCTCATATGCGTTCTGTGAT |
| | hypothetical | VSA | TGTTGCCTTCCTGTTTGCCTGT |
| | protein | (SEQ ID NO: 15) | AGCTTTTGCGTGAGCGCCGGCG |
| | (WP_001258047) | | AGAGACC |
| | signal | | (SEQ ID NO: 6) |
| | sequence | | |
| L4 | E. coli OmpT | MRAKLLGIVLTTPIA | GGTCTCATATGCGTGCGAAACT |
| | signal | ISSFA | GCTCGGCATTGTTCTGACCACC |
| | sequence | (SEQ ID NO: 16) | CCGATTGCCATTTCCAGCTTTG |
| | | | CCGGCGAGAGACC |
| | | | (SEQ ID NO: 7) |
| L5 | E. coli OmpA | MKKTAIAIAVALAGF | GGTCTCATATGAAAAAAACCGC |
| | signal | ATVAQA | CATCGCCATTGCCGTTGCCCTC |
| | sequence | (SEQ ID NO: 17) | GCTGGCTTTGCCACCGTGGCGC |
| | | | AGGCGGGCGAGAGACC |
| | | | (SEQ ID NO: 8) |
| L6 | M13 G4 signal | MKLLNVINFVFLMFV | GGTCTCATATGAAACTGCTGAA |
| | sequence | SSSSFA | CGTGATCAACTTTGTTTTCCTG |
| | | (SEQ ID NO: 18) | ATGTTTGTCAGCAGCAGTAGTT |
| | | | TTGCCGGCGAGAGACC |
| | | | (SEQ ID NO: 9) |
| L7 | M13 G3 signal | MKKLLFAIPLVVPFY | GGTCTCATATGAAAAAACTGCT |
| | sequence | SHS | GTTTGCCATTCCGCTGGTTGTA |
| | | (SEQ ID NO: 19) | CCGTTTTACAGCCACAGCGGCG |
| | | | AGAGACC |
| | | | (SEQ ID NO: 10) |
| L8 | E. coli Lpp | MKATKLVLGAVILGS | GGTCTCATATGAAAGCGACGAA |
| | signal | TLLAG | ACTGGTGCTGGGTGCTGTGATT |
| | sequence | (SEQ ID NO: 20) | CTGGGCAGCACGCTGCTGGCGG |
| | | | GCGGCGAGAGACC |
| | | | (SEQ ID NO: 11) |
| L9 | E. coli GspD | MKGLNKITCCLLAAL | GGTCTCATATGAAAGGTCTGAA |
| | signal | LMPCAGHA | TAAAATTACCTGCTGTTTACTG |
| | sequence | (SEQ ID NO: 21) | GCGGCGCTGCTGATGCCGTGCG |
| | | | CGGGTCATGCGGGCGAGAGACC |
| | | | (SEQ ID NO: 12) |

### Example 1.4: Construction of Plasmid for CRM197 Overexpression

Plasmids for overexpressing CRM197 in E. coli and secreting the CRM197 into the periplasm were prepared as shown in FIG. 3. After double digestion of pHex1.3 with BsaI and DraI, a DNA fragment about 5 kb long was isolated using agarose electrophoresis. The signal sequences L1 to L9 were amplified by PCR using the primers and templates shown in Table 1. The CRM197 DNA fragment to fuse the CRM197 gene with each signal sequence using the LIC method was amplified by PCR using the primers and templates shown in Table 1. pHex1.3 digested with BsaI and DraI, each signal sequence fragment, and a CRM197 fragment compatible therewith were assembled in vitro using the LIC conditions described above, and then the resulting product was transformed into E. coli C2984H to prepare plasmids pHex-L1-CRM, pHex-L2-CRM, pHex-L3-CRM, pHex-L4-CRM, pHex-L5-CRM, pHex-L6-CRM, pHex-L7-CRM, pHex-L8-CRM and pHex-L9-CRM comprising CRM197 fused with respective signal sequences.

### EXAMPLE 2: EXPRESSION OF CRM197 PROTEIN IN E. COLI

pHex-L3-CRM and pHex-L5-CRM were selected from the plasmids prepared in Example 1, in consideration of the expression level and the degree of cell growth. After transformation of pHex-L3-CRM and pHex-L5-CRM into the E. coli strain of Table 4 below, the expression and expression position of the CRM197 protein were evaluated.

The culture method is as follows. The colony produced on a solid medium (10 g/L Soytone, 5 g/L yeast extract, 10 g/L NaCl, 15 g/L agar) was shaking-cultured in LB liquid medium containing 100 mM potassium phosphate (pH 7.5), km 50 μg/ml and 0.2% lactose, all cells were subjected to SDS-PAGE, and then CRM197 expression was analyzed using Coomassie staining and Western blotting (FIG. 4). For the L3 fusion, CRM197 protein was expressed in all strains used, whereas for the L5 fusion, growth was impossible in liquid medium when transformed into Origami™ 2. In other strains, CRM197 protein expression was observed.

TABLE 4
E. coli strains used in the present invention

| | | Vendor | | Genotype |
|---|---|---|---|---|
| O | Origami ™ 2 | Novagen | K12 | Δ(ara-leu)7697 ΔlacX74 |
| | | | | ΔphoAPvuIIphoR araD139 |
| | | | | ahpCgalEgalKrpsL F′[lac+ lacIq |
| | | | | pro] gor522::Tn10 trxB(CamR, |
| | | | | StrR, TetR) |
| S | Shuffle | NEB | B | fhuA2 [lon] ompTahpC gal |
| | | | | λatt::pNEB3-r1-cDsbC (SpecR, |
| | | | | lacIq) ΔtrxBsulA11 R(mcr- |
| | | | | 73::miniTn10--TetS)2 [dcm] R(zgb- |
| | | | | 210::Tn10 --TetS) endA1 Δgor |
| | | | | Δ(mcrC-mrr)114::IS10 |
| C | C2894H | NEB | K12 | F′ proA+B+ lacIq ΔlacZM15/ |
| | | | | fhuA2 Δ(lac-proAB) glnV |
| | | | | galK16 galE15 |
| | | | | R(zgb-210::Tn10)TetS endA1 |
| | | | | thi-1 Δ(hsdS-mcrB) 5 |
| W | W3110-1 | KCTC | K12 | F-λ-rph-1 INV(rrnD, rrnE), |
| | | | | ΔompT |
| B | BL21 (DE3) | | B | F-ompT gal dcmlonhsdSB |
| | | | | (rB-mB-) λ(DE3 |
| | | | | [lacI lacUV5-T7p07 ind1 sam7 |
| | | | | nin5]) [malB+]K-12(λS) |

### Example 2.1: CRM197 Expression by L5

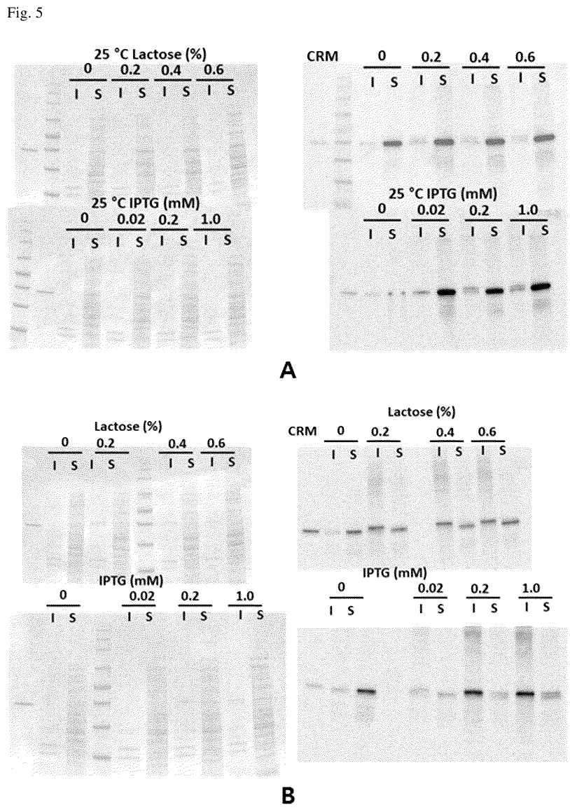

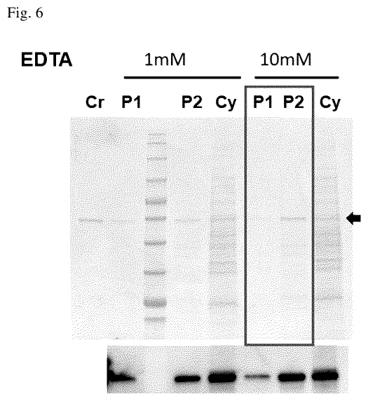

BL21 (DE3) containing pHex-L5-CRM was cultured in LB liquid medium (50 mL/500 mL baffled flask) containing 100 mM potassium phosphate (pH 7.5) and km 50 μg/ml until OD₆₀₀ reached 0.4 to 0.6. Then, 0.2, 0.4, or 0.6% lactose or 0.02, 0.2, or 2 mM IPTG (isopropyl β-D-1-thiogalactopyanosid) was added as an inducer to induce expression. The culture temperature was 25° C. or 30° C. After culturing, the cells were recovered, suspended in 50 mM potassium phosphate (pH 7.0), and then disrupted by sonication. After disruption, centrifugation was performed to separate the supernatant (soluble fraction) from the precipitate (insoluble fraction). After SDS-PAGE of each sample, expression of the CRM197 protein was analyzed using Coomassie staining and Western blotting (FIG. 5). When the inducer (lactose, IPTG) was added and culture was then performed at 25° C., most of the expressed CRM197 protein was present in a soluble form, and had the same molecular weight of the reference CRM197, which indicates that the expressed protein was mature CRM197 from which the L5 signal sequence was removed (FIG. 5A). On the other hand, when lactose was used as the inducer and culture was performed at 30° C., about 50% of the CRM197 protein was found in the insoluble fraction, and when IPTG was added as the inducer, most of the CRM197 protein was found in the insoluble fraction (FIG. 5B).

The periplasm fraction was recovered using osmotic shock to detect the location at which CRM197 was expressed. The process is as follows. BL21 (DE3) containing pHex-L5-CRM was cultured at 25° C. and then centrifuged to recover cells. The cells were resuspended in plasma membrane induction buffer [30 mM Tris-HCl (pH 8.0), 20% sucrose, 1 or 10 mM EDTA, 1 mM PMSF (phenylmethylsulfonyl fluoride)] until the cell concentration reached OD₆₀₀ of 10 and stirred at room temperature for 0.5 to 1 hour. Then, the cells were collected by centrifugation at 4,000×g for 15 minutes and added with the same amount of 30 mM cold (4° C. or lower) Tris-HCl (pH 8.0), followed by stirring at room temperature for 0.5 to 1 hour. Then, the cells were centrifuged at 4,000×g for 15 minutes to obtain the supernatant (periplasmic fraction, P2). After treatment with plasma membrane induction buffer, the supernatant (P1), periplasmic fraction (P2), and cytoplasm fraction were developed using SDS-PAGE, and then the expression of CRM197 and location of the expressed CRM197 were evaluated using Coomassie staining and Western blotting (FIG. 5). It was found that L5 can successfully secrete CRM197 into the periplasm (FIG. 6). EDTA as plasma membrane induction buffer was more effective at 10 mM than 1 mM.

### Example 2.2: CRM197 Expression by L3

BL21 (DE3) containing pHex-L3-CRM was expressed under the same conditions as in Example 2.1. It was found that the CRM197 induced by L3, unlike L5, was present in a soluble form under all conditions (FIG. 7), and was secreted into the periplasm (FIG. 8).

### EXAMPLE 3: CULTURE of E. COLI BL21 (DE3)

The BL21 (DE3) strain containing pHex-L3-CRM or pHex-L5-CRM was cultured using the following method. During main culture, feeding was performed using a pH stat method, and the pH was maintained at 7.3 using a feeding solution (600 g/L glucose, 30 g/L yeast extract) and an alkali solution (14-15% ammonia). The compositions of solutions and media used for culture are shown in Tables 5 and 6.

TABLE 5
Composition of solutions and media used for culture in
present invention

| | | Seed | | Main | | | |
|---|---|---|---|---|---|---|---|
| | | culture | | culture | | Feeding | |
| | | medium | | medium | | solution | pH |
| | | (/L) | | (/L) | | (/L) | adjustment |
| Casamino | | 20 | g | 20 | g | — | Using |
| acids | | | | | | | ammonia |
| Yeast | | 10 | g | 10 | g | 30 g | solution |
| extract | | | | | | | (14~15%) |
| (NH₄)₂SO₄ | | 7 | g | 7 | g | — | |
| K₂HPO₄ | | 2.5 | g | 2.5 | g | — | |
| NaCl | | 0.5 | g | 0.5 | g | — | |
| Trace | | 10 | mL | 10 | mL | — | |
| metal | | | | | | | |
| (100x) | | | | | | | |
| Glutamic | Added | 2 | g | 2 | g | — | |
| acids | after | | | | | | |
| CaCl₂•2H₂O | auto- | 10 | mg | 10 | mg | — | |
| Glucose | clave | 15 | g | 15 | g | 600 g | |
| MgSO₄•7H₂O | | 2.5 | g | 2.5 | g | — | |
| Km50 | | 1 | mL | 1 | mL | — | |
| SB2121 | | — | | 0.1% | | — | |
| (anti foam) | | | | | | | |

pH adjustment to 7.3
(using 1~4M NaOH)

TABLE 6
Composition of trace metal used for
culture of present invention

| | Trace metal (100x, /L) | |
|---|---|---|
| EDTA | 840 | mg |
| CoCl₂•6H2O | 250 | mg |
| MnCl₂•4H₂O | 1.5 | g |
| CuC1₂•2H₂O | 150 | mg |
| H₃BO₃ | 300 | mg |
| Na₂MoO₄•2H₂O | 250 | mg |
| Zn(CH₃COO)₂•2H₂O | 1.3 | g |
| Fe (III) citrate | 10 | g |

A single colony formed in modified LB agar medium [modified Luria-Bertani (LB) agar: 10 g/L soytone, 5 g/L yeast extract, 10 g/L sodium chloride, 15 g/L agar, 50 mg/L kanamycin] was inoculated into a seed culture medium, followed by incubation at 30° C. for 18 hours. The resulting seed culture was again inoculated at a ratio of 1% (v/v) in a main culture medium (3L/5L fermenter) and cultured at 30° C. The main culture medium was obtained by adding 0.1% of a sterilized antifoaming agent to the seed culture medium. After the absorbance of the culture solution reached 30-40, the temperature was lowered to 25° C. Then, 10 mM IPTG was added and the culture was terminated after the absorbance of the culture solution reached 100˜120.

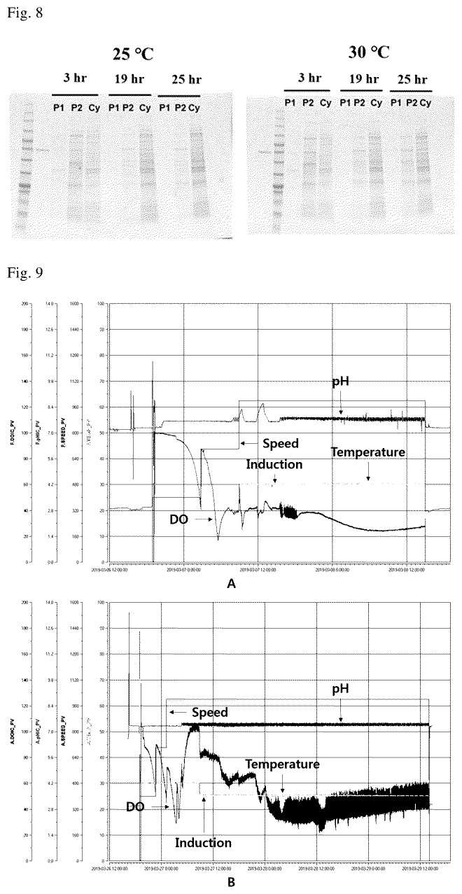

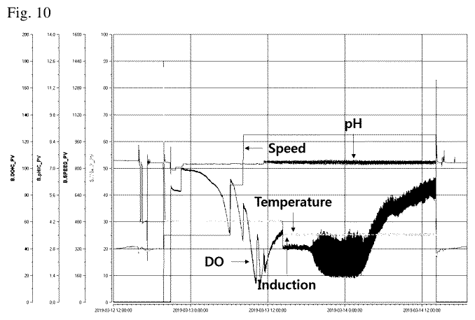

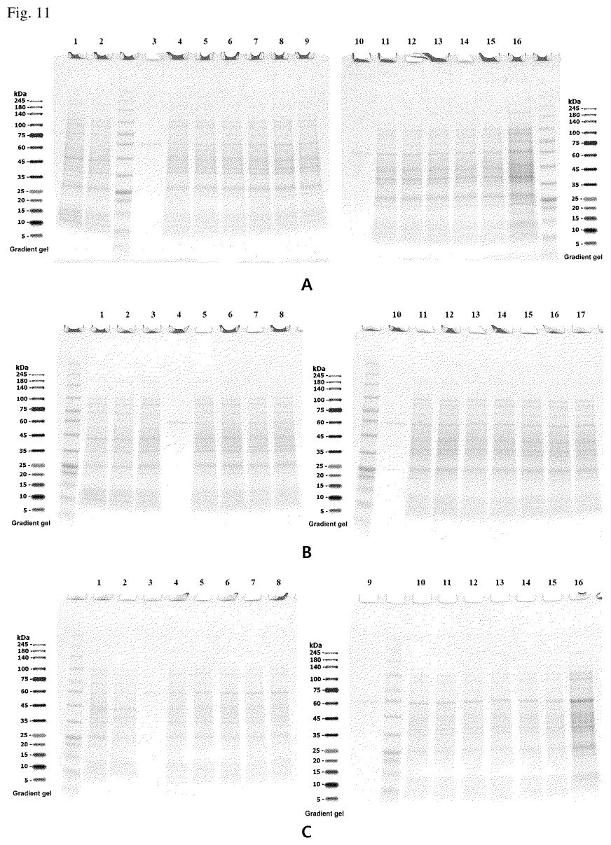

The culture behavior of E. coli BL21 (DE3) containing pHex-L3-CRM in a 5L fermenter is shown in FIG. 9, and the culture behavior of E. coli BL21 (DE3) containing pHex-L5-CRM is shown in FIG. 10. The expression behavior of CRM197 upon fermentation in a 5L fermenter is shown in FIG. 11, and the expression yield was 1.1 to 1.2 g/L for L3 fusion and 3.0 to 3.7 g/L for L5 fusion. The quantity of CRM197 was measured by comparing the relative quantity with reference CRM197 using a densitometer (GS-900™, Bio-Rad laboratories Ins., Hercules, California) after SDS-PAGE/Coomassie staining.

### EXAMPLE 4: PROTEIN PURIFICATION

Example 4.1: Production of Periplasmic Fraction from Cell Culture

The procedure for recovering the periplasmic fraction from cells cultured in a 5L fermenter is as follows. The cell culture medium was centrifuged at 4° C. and 4,000×g for 15 minutes to precipitate cells. The cell precipitate was resuspended in a plasma membrane induction buffer (Table 7) of protein modified based on an absorbance of 100 and stirred at room temperature for 0.5 to 1 hour.

TABLE 7
Composition of buffer solution used for preparation
of periplasmic fraction of present invention

| | Periplasting | Shock buffer |
|---|---|---|
| | buffer | (cold) |
| Tris-HCl (pH 8.0) | 30 mM | 30 mM |
| Sucrose | 20% | — |
| EDTA | 10 mM | — |
| PMSF | 1 mM | — |

Then, the cells were collected by centrifugation at 4,000×g for 15 minutes and were added with the same amount of 30 mM cold (at 4° C. or less) Tris-HCl (pH 8.0), followed by stirring at room temperature for 0.5 to 1 hour. Then, the cells were centrifuged at 4,000×g for 15 minutes to obtain the supernatant, and impurities were removed using MF. SDS-PAGE analysis of the periplasmic fraction recovered from E. coli BL21 (DE3) containing pHex-L3-CRM through the process described above is shown in FIG. 12(A), and SDS-PAGE analysis of the periplasmic fraction recovered from E. coli BL21 (DE3) containing pHex-L5-CRM is shown in FIG. 12(B). The amount of CRM197 protein present in the periplasmic fraction was found to be 1.2 g/L for the L3 strain and 2.3 g/L for the L5 strain (Table 8).

TABLE 8
Amount of CRM197 protein obtained through culture of present invention

| | L3 | | L5 | | | Periplasm | |
|---|---|---|---|---|---|---|---|
| | batch #1 | batch #2 | batch #1 | batch #2 | batch #3 | L3_#2 | L5_#1 |
| Final | 109.4 | 118 | 119.8 | 107 | 112 | 100 | 100 |
| OD₆₀₀ | | | | | | | |
| Total CRM | 1.18 | 1.06 | 3.74 | 2.95 | 3.74 | 1.18 | 2.30 |
| (g/L) | | | | | | | |

### Example 4.2: Purification of CRM197 Protein

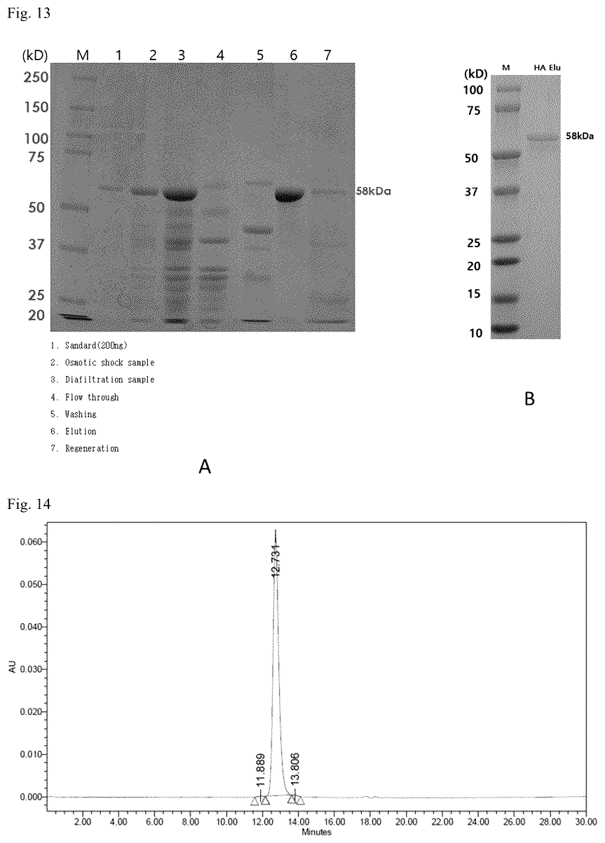

The periplasmic fraction of the pHex-L3-CRM culture medium was concentrated twice with a 10 kDa cut-off membrane using a TFF system, and ultrafiltration was performed using ten volumes of 10 mM sodium phosphate solvent (pH 7.2). Purification was completed through two column processes using an AKTA pure (GE Healthcare) system. The first column process was anion exchange chromatography (diethyl aminoethyl Sepharose fast flow resin, DEAE), and was used to remove nucleic acids and impurity proteins. The DEAE resin is negatively charged (−) and is bound with a positively charged (+) protein. Unbound proteins and impurities were primarily extracted and removed from the ultra-filtered sample through DEAE chromatography, and then impure proteins with low binding ability, excluding CRM197, were removed through a subsequent washing process based on the salt concentration. Then, only CRM197 was eluted by increasing the salt concentration. SDS-PAGE analysis was performed on the sample during the purification process using DEAE chromatography, and the results are shown in FIG. 13(A). First, unbound impurities and proteins were primarily removed in a flow-through manner from the sample subjected to DEAE chromatography using hydroxyapatite (HA) chromatography, and CRM197 was eluted with 100 mM potassium phosphate and 100 mM NaCl solvents. The result of SDS-PAGE of the eluted CRM197 is shown in FIG. 13(B).

### EXAMPLE 5: COMPARISON WITH CRM197 PRODUCED USING CORYNEBACTERIUM

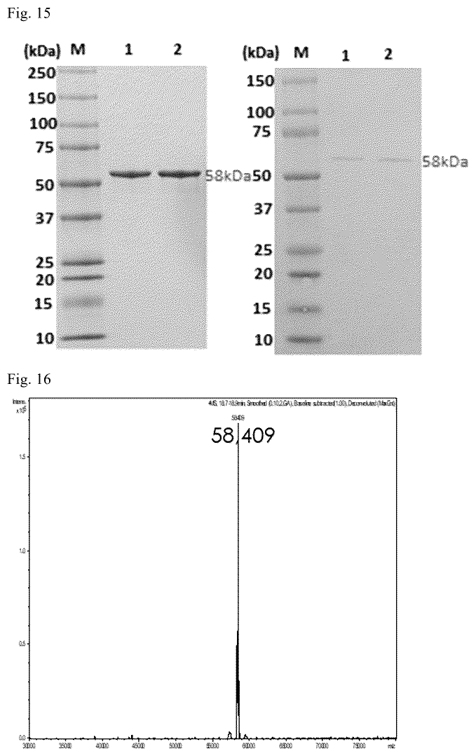

The quality and characteristics of the final purified CRM197 were analyzed. As a result of SEC-HPLC analysis, it was found that the purity was 99% or more (FIG. 14). SDS-PAGE and Western blotting analysis showed that a band appeared at the same position as that of CRM197 produced using Corynebacterium (FIG. 15).

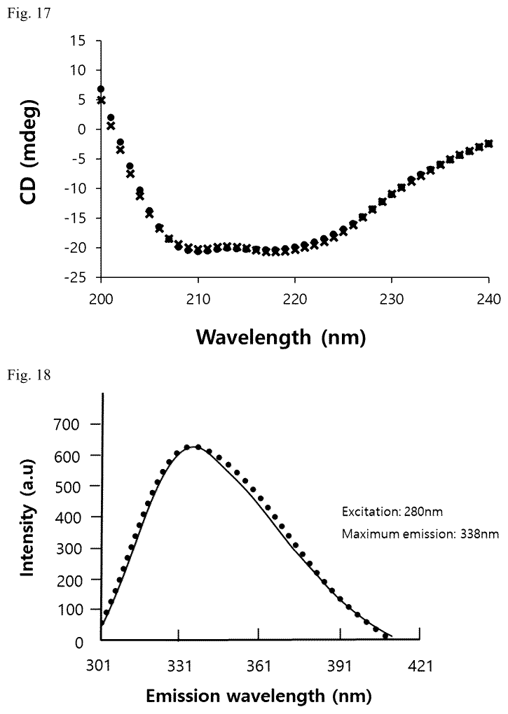

In addition, the entire sequence of 535 amino acids constituting CRM197 was found to be 100% identical, and as a result of molecular weight measurement, a main peak of 58,409 Da was identified, which corresponded to the theoretical molecular weight (FIG. 16). The higher-order structure was identified through circular dichroism (CD) analysis, and it was found that there was no difference in the higher-order structure with the CRM197 produced using Corynebacterium (FIG. 17). Fluorescence spectrum analysis showed that the maximum emission wavelength was 338 nm, which was the same as that of the CRM197 produced using Corynebacterium (FIG. 18).

The results described above showed that CRM197 produced using the E. coli pHex-L3strain was physicochemically and immunologically the same as the CRM197 produced using Corynebacterium.

### INDUSTRIAL APPLICABILITY

The present invention is very useful for CRM197 protein production because CRM197protein having the same physicochemical/immunological properties as the protein isolated from the parent bacteria can be expressed in general E. coli in which the redox potential is not regulated, and CRM197 protein having high secretion efficiency into the periplasm can be produced without a shift of pH of the medium for increasing secretion into the periplasm.

Although specific configurations of the present invention have been described in detail, those skilled in the art will appreciate that this description is provided to set forth preferred embodiments for illustrative purposes and should not be construed as limiting the scope of the present invention. Therefore, the substantial scope of the present invention is defined by the accompanying claims and equivalents thereto.

SEQUENCE LISTING FREE TEXT

An electronic file is attached.

What is claimed is:

1. A nucleic acid comprising a nucleotide sequence encoding a signal sequence for expressing a CRM197 protein, wherein the nucleotide sequence is SEQ ID NO: 6.

2. A nucleic acid construct comprising the nucleic acid according to claim 1 and a gene of the CRM197 protein.

3. The nucleic acid construct according to claim 2, wherein the gene of the CRM197 protein comprises the nucleotide sequence of SEQ ID NO: 2.

4. An expression vector comprising the nucleic acid according to claim 1 and a gene of the CRM197 protein.

5. The expression vector according to claim 4, wherein the gene of the CRM197 protein comprises the nucleotide sequence of SEQ ID NO: 2.

6. The expression vector according to claim 4, further comprising a Trc promotor.

7. A recombinant microorganism, wherein the recombinant microorganism is transformed with the nucleic acid construct according to claim 2 or is transformed with an expression vector comprising said nucleic acid construct.

8. The recombinant microorganism according to claim 7, wherein the recombinant microorganism is Escherichia coli.

9. A method for producing a CRM197 protein comprising:
(a) culturing the recombinant microorganism according to claim 7 to produce a CRM197 protein; and
(b) recovering the produced CRM197 protein.

10. The method according to claim 9, wherein the step (b) comprises recovering the CRM197 protein secreted into the periplasm.


SEQUENCE LISTING (56 sequences)

SEQ ID NO: 1 - DNA, 1226 bp, synthetic construct
  misc_feature 1..1226: note = Synthetic construct
  source 1..1226: mol_type = other DNA
tactgagctaataacaggcctgctggtaatcgcaggcctttttatttctggctcaccttcgggtgggcctttctgcgtttacggttctggcaaatattctgaaatgagctgttgacaattaatcatccggctcgtataatgtgtggaattgtgagcggataacaatttactgctctttaacaatttatcagatccaattggaggaacaatatgggagaccacgcgcgcgaggtctcatctgcggcggcagcagggggatcgatgagcaaaggtgaagagctgtttaccggtgtggtgccgattctggttgaactggatggcgacgttaacggccataaattcagcgtcagcggcgagggcgaaggggatgccacctacggtaaactgaccctgaagtttatttgcaccaccggcaaattaccggttccgtggccgacgctggtgacgacctttagctatggcgtgcagtgcttttcccgttatccggaccatatgaaacagcatgatttttttaaaagcgcgatgccggaaggctatgttcaggaacgtaccattttctttaaggatgacggcaattacaaaacccgcgcggaagtgaaatttgaaggtgataccctggtcaaccgcattgaactgaaaggcattgatttcaaagaagatggtaatattctcggtcataagctggaatataactacaacagccataacgtttatatcatggcggataaacaaaaaaacggtattaaagtgaactttaaaattcgccataatatcgaagatggtagcgtgcaactggcggatcattatcagcagaacaccccaattggcgatggcccggtgttgctgccggataaccactatctgagcacccagtcggcgctgagtaaagatccgaacgaaaaacgcgatcacatggtgctgctggagtttgtcaccgccgccggtatcacccacggcatggatgaactgtataaataatgatttaaagcggatatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtt

SEQ ID NO: 2 - DNA, 1625 bp, synthetic construct
  misc_feature 1..1625: note = Synthetic construct
  source 1..1625: mol_type = other DNA
gaagacacggcgctgacgatgttgttgacagcagcaagagctttgttatggagaatttcagcagctatcacggcaccaaaccgggctatgttgacagcattcagaaaggcatccaaaagccgaaaagcggtacccagggcaactacgacgatgactggaaagagttttatagcaccgataacaagtacgacgcggcgggctatagcgttgataacgaaaacccgctgagcggtaaagcgggtggcgtggttaaggtgacctacccgggtctgaccaaagtgctggcgctgaaggttgacaacgcggaaaccatcaagaaagagctgggcctgagcctgaccgaaccgctgatggagcaggttggtaccgaggaattcattaagcgttttggtgatggtgcgagccgtgtggttctgagcctgccgttcgcggaaggtagcagcagcgtggagtacatcaacaactgggaacaagcgaaagcgctgagcgtggagctggaaattaacttcgaaacccgtggcaagcgtggccaggatgcgatgtacgaatatatggcgcaagcgtgcgcgggtaaccgtgtgcgtcgtagcgttggtagcagcctgagctgcattaacctggattgggacgttatccgtgacaagaccaaaaccaagatcgaaagcctgaaagagcacggtccgattaaaaacaagatgagcgagagcccgaacaagaccgtgagcgaggaaaaagcgaagcagtacctggaggaatttcaccaaaccgcgctggagcacccggaactgagcgagctgaaaaccgtgaccggcaccaacccggttttcgcgggtgcgaactatgcggcgtgggcggtgaacgttgcgcaggtgatcgatagcgaaaccgcggacaacctggaaaagaccaccgcggcgctgagcatcctgccgggtattggcagcgtgatgggcatcgcggatggtgcggttcaccacaacaccgaggaaatcgtggcgcagagcattgcgctgagcagcctgatggttgcgcaagcgatcccgctggttggtgagctggttgacattggtttcgcggcgtacaactttgtggaaagcatcattaacctgttccaggtggttcacaacagctacaaccgtccggcgtatagcccgggccacaaaacccaaccgtttctgcacgatggttatgcggtgagctggaacaccgttgaggacagcatcattcgtaccggtttccagggcgagagcggtcacgatatcaagattaccgcggaaaacaccccgctgccgattgcgggcgttctgctgccgaccattccgggtaaactggacgttaacaaaagcaagacccacattagcgtgaacggccgtaagatccgtatgcgttgccgtgcgattgatggtgacgtgaccttttgccgtccgaaaagcccggtgtacgttggtaacggcgtgcacgcgaacctgcacgttgcgttccaccgtagcagcagcgagaagatccacagcaacgaaattagcagcgacagcatcggcgttctgggttatcaaaaaaccgtggatcataccaaagtgaatagcaagctgagcctgttcttcgagattaaaagctctgaggtcttc

SEQ ID NO: 3 - protein, 535 aa, synthetic construct
  REGION 1..535: note = Synthetic construct
GADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 4 - DNA, 85 bp, synthetic construct
  misc_feature 1..85: note = Synthetic construct
  source 1..85: mol_type = other DNA
ggtctcatatgaaatatctgttaccgaccgccgctgccggactgctgttactggcggcgcagccggcgatggcgggcgagagacc

SEQ ID NO: 5 - DNA, 88 bp, synthetic construct
  misc_feature 1..88: note = Synthetic construct
  source 1..88: mol_type = other DNA
ggtctcatatgaaaaaaagcctggttctgaaagcgtctgttgcggtggcgacgctggtgccgatgctgtcgtttgccggcgagagacc

SEQ ID NO: 6 - DNA, 73 bp, synthetic construct
  misc_feature 1..73: note = Synthetic construct
  source 1..73: mol_type = other DNA
ggtctcatatgcgttctgtgattgttgccttcctgtttgcctgtagcttttgcgtgagcgccggcgagagacc

SEQ ID NO: 7 - DNA, 79 bp, synthetic construct
  misc_feature 1..79: note = Synthetic construct
  source 1..79: mol_type = other DNA
ggtctcatatgcgtgcgaaactgctcggcattgttctgaccaccccgattgccatttccagctttgccggcgagagacc

SEQ ID NO: 8 - DNA, 82 bp, synthetic construct
  misc_feature 1..82: note = Synthetic construct
  source 1..82: mol_type = other DNA
ggtctcatatgaaaaaaaccgccatcgccattgccgttgccctcgctggctttgccaccgtggcgcaggcgggcgagagacc

SEQ ID NO: 9 - DNA, 82 bp, synthetic construct
  misc_feature 1..82: note = Synthetic construct
  source 1..82: mol_type = other DNA
ggtctcatatgaaactgctgaacgtgatcaactttgttttcctgatgtttgtcagcagcagtagttttgccggcgagagacc

SEQ ID NO: 10 - DNA, 73 bp, synthetic construct
  misc_feature 1..73: note = Synthetic construct
  source 1..73: mol_type = other DNA
ggtctcatatgaaaaaactgctgtttgccattccgctggttgtaccgttttacagccacagcggcgagagacc

SEQ ID NO: 11 - DNA, 79 bp, synthetic construct
  misc_feature 1..79: note = Synthetic construct
  source 1..79: mol_type = other DNA
ggtctcatatgaaagcgacgaaactggtgctgggtgctgtgattctgggcagcacgctgctggcgggcggcgagagacc

SEQ ID NO: 12 - DNA, 88 bp, synthetic construct
  misc_feature 1..88: note = Synthetic construct
  source 1..88: mol_type = other DNA
ggtctcatatgaaaggtctgaataaaattacctgctgtttactggcggcgctgctgatgccgtgcgcgggtcatgcgggcgagagacc

SEQ ID NO: 13 - protein, 22 aa, synthetic construct
  REGION 1..22: note = Synthetic construct
MKYLLPTAAAGLLLLAAQPAMA

SEQ ID NO: 14 - protein, 23 aa, synthetic construct
  REGION 1..23: note = Synthetic construct
MKKSLVLKASVAVATLVPMLSFA

SEQ ID NO: 15 - protein, 18 aa, synthetic construct
  REGION 1..18: note = Synthetic construct
MRSVIVAFLFACSFCVSA

SEQ ID NO: 16 - protein, 20 aa, synthetic construct
  REGION 1..20: note = Synthetic construct
MRAKLLGIVLTTPIAISSFA

SEQ ID NO: 17 - protein, 21 aa, synthetic construct
  REGION 1..21: note = Synthetic construct
MKKTAIAIAVALAGFATVAQA

SEQ ID NO: 18 - protein, 21 aa, synthetic construct
  REGION 1..21: note = Synthetic construct
MKLLNVINFVFLMFVSSSSFA

SEQ ID NO: 19 - protein, 18 aa, synthetic construct
  REGION 1..18: note = Synthetic construct
MKKLLFAIPLVVPFYSHS

SEQ ID NO: 20 - protein, 20 aa, synthetic construct
  REGION 1..20: note = Synthetic construct
MKATKLVLGAVILGSTLLAG

SEQ ID NO: 21 - protein, 23 aa, synthetic construct
  REGION 1..23: note = Synthetic construct
MKGLNKITCCLLAALLMPCAGHA

SEQ ID NO: 22 - DNA, 34 bp, synthetic construct
  misc_feature 1..34: note = Synthetic construct
  source 1..34: mol_type = other DNA
gcggatccaagagacaggatgaggatcgtttcgc

SEQ ID NO: 23 - DNA, 40 bp, synthetic construct
  misc_feature 1..40: note = Synthetic construct
  source 1..40: mol_type = other DNA
cggatatcaagcttggaaatgttgaatactcatactcttc

SEQ ID NO: 24 - DNA, 30 bp, synthetic construct
  misc_feature 1..30: note = Synthetic construct
  source 1..30: mol_type = other DNA
gagatccggagcttatactgagctaataac

SEQ ID NO: 25 - DNA, 29 bp, synthetic construct
  misc_feature 1..29: note = Synthetic construct
  source 1..29: mol_type = other DNA
gaaaaataaacaaaaacaaaaagagtttg

SEQ ID NO: 26 - DNA, 31 bp, synthetic construct
  misc_feature 1..31: note = Synthetic construct
  source 1..31: mol_type = other DNA
tacaaactctttttgtttttgtttatttttc

SEQ ID NO: 27 - DNA, 34 bp, synthetic construct
  misc_feature 1..34: note = Synthetic construct
  source 1..34: mol_type = other DNA
cctgttattagctcagtataagctccggatctcg

SEQ ID NO: 28 - DNA, 26 bp, synthetic construct
  misc_feature 1..26: note = Synthetic construct
  source 1..26: mol_type = other DNA
aattggaggaacaatatgaaatatct

SEQ ID NO: 29 - DNA, 29 bp, synthetic construct
  misc_feature 1..29: note = Synthetic construct
  source 1..29: mol_type = other DNA
aacatcgtcagcgcccgccatcgccggct

SEQ ID NO: 30 - DNA, 27 bp, synthetic construct
  misc_feature 1..27: note = Synthetic construct
  source 1..27: mol_type = other DNA
ttggaggaacaatatgaaaaaaagcct

SEQ ID NO: 31 - DNA, 30 bp, synthetic construct
  misc_feature 1..30: note = Synthetic construct
  source 1..30: mol_type = other DNA
aacatcgtcagcgccggcaaacgacagcat

SEQ ID NO: 32 - DNA, 26 bp, synthetic construct
  misc_feature 1..26: note = Synthetic construct
  source 1..26: mol_type = other DNA
ttggaggaacaatatgcgttctgtga

SEQ ID NO: 33 - DNA, 28 bp, synthetic construct
  misc_feature 1..28: note = Synthetic construct
  source 1..28: mol_type = other DNA
aacatcgtcagcgccggcgctcacgcaa

SEQ ID NO: 34 - DNA, 27 bp, synthetic construct
  misc_feature 1..27: note = Synthetic construct
  source 1..27: mol_type = other DNA
ttggaggaacaatatgcgtgcgaaact

SEQ ID NO: 35 - DNA, 30 bp, synthetic construct
  misc_feature 1..30: note = Synthetic construct
  source 1..30: mol_type = other DNA
aacatcgtcagcgccggcaaagctggaaat

SEQ ID NO: 36 - DNA, 25 bp, synthetic construct
  misc_feature 1..25: note = Synthetic construct
  source 1..25: mol_type = other DNA
ttggaggaacaatatgaaaaaaacc

SEQ ID NO: 37 - DNA, 30 bp, synthetic construct
  misc_feature 1..30: note = Synthetic construct
  source 1..30: mol_type = other DNA
aacatcgtcagcgcccgcctgcgccacggt

SEQ ID NO: 38 - DNA, 26 bp, synthetic construct
  misc_feature 1..26: note = Synthetic construct
  source 1..26: mol_type = other DNA
ttggaggaacaatatgaaactgctga

SEQ ID NO: 39 - DNA, 30 bp, synthetic construct
  misc_feature 1..30: note = Synthetic construct
  source 1..30: mol_type = other DNA
aacatcgtcagcgccggcaaaactactgct

SEQ ID NO: 40 - DNA, 25 bp, synthetic construct
  misc_feature 1..25: note = Synthetic construct
  source 1..25: mol_type = other DNA
ttggaggaacaatatgaaaaaactg

SEQ ID NO: 41 - DNA, 29 bp, synthetic construct
  misc_feature 1..29: note = Synthetic construct
  source 1..29: mol_type = other DNA
acatcgtcagcgccgctgtggctgtaaaa

SEQ ID NO: 42 - DNA, 28 bp, synthetic construct
  misc_feature 1..28: note = Synthetic construct
  source 1..28: mol_type = other DNA
ttggaggaacaatatgaaagcgacgaaa

SEQ ID NO: 43 - DNA, 30 bp, synthetic construct
  misc_feature 1..30: note = Synthetic construct
  source 1..30: mol_type = other DNA
aacatcgtcagcgccgcccgccagcagcgt

SEQ ID NO: 44 - DNA, 27 bp, synthetic construct
  misc_feature 1..27: note = Synthetic construct
  source 1..27: mol_type = other DNA
ttggaggaacaatatgaaaggtctgaa

SEQ ID NO: 45 - DNA, 30 bp, synthetic construct
  misc_feature 1..30: note = Synthetic construct
  source 1..30: mol_type = other DNA
aacatcgtcagcgcccgcatgacccgcgca

SEQ ID NO: 46 - DNA, 27 bp, synthetic construct
  misc_feature 1..27: note = Synthetic construct
  source 1..27: mol_type = other DNA
agccggcgatggcgggcgctgacgatg

SEQ ID NO: 47 - DNA, 28 bp, synthetic construct
  misc_feature 1..28: note = Synthetic construct
  source 1..28: mol_type = other DNA
atgctgtcgtttgccggcgctgacgatg

SEQ ID NO: 48 - DNA, 26 bp, synthetic construct
  misc_feature 1..26: note = Synthetic construct
  source 1..26: mol_type = other DNA
ttgcgtgagcgccggcgctgacgatg

SEQ ID NO: 49 - DNA, 27 bp, synthetic construct
  misc_feature 1..27: note = Synthetic construct
  source 1..27: mol_type = other DNA
tttccagctttgccggcgctgacgatg

SEQ ID NO: 50 - DNA, 28 bp, synthetic construct
  misc_feature 1..28: note = Synthetic construct
  source 1..28: mol_type = other DNA
accgtggcgcaggcgggcgctgacgatg

SEQ ID NO: 51 - DNA, 28 bp, synthetic construct
  misc_feature 1..28: note = Synthetic construct
  source 1..28: mol_type = other DNA
agcagtagttttgccggcgctgacgatg

SEQ ID NO: 52 - DNA, 28 bp, synthetic construct
  misc_feature 1..28: note = Synthetic construct
  source 1..28: mol_type = other DNA
ttttacagccacagcggcgctgacgatg

SEQ ID NO: 53 - DNA, 24 bp, synthetic construct
  misc_feature 1..24: note = Synthetic construct
  source 1..24: mol_type = other DNA
tgctggcgggcggcgctgacgatg

SEQ ID NO: 54 - DNA, 26 bp, synthetic construct
  misc_feature 1..26: note = Synthetic construct
  source 1..26: mol_type = other DNA
cgcgggtcatgcgggcgctgacgatg

SEQ ID NO: 55 - DNA, 36 bp, synthetic construct
  misc_feature 1..36: note = Synthetic construct
  source 1..36: mol_type = other DNA
gatatccgcttttcattagcttttaatctcgaagaa

SEQ ID NO: 56 - DNA, 30 bp, synthetic construct
  misc_feature 1..30: note = Synthetic construct
  source 1..30: mol_type = other DNA
ggcgcaagcgtgcgcgggtaaccgtgtgcg